(19) United States
(12) Patent Application Publication
(10) Pub. No.: US 2016/0074552 A1
(43) Pub. Date: Mar. 17, 2016

(54) A DISCONTINUOUS HYDROCOLLOID ARTICLE

(71) Applicant: 3M INNOVATIVE PROPERTIES COMPANY, Saint Paul, MN (US)

(72) Inventors: JUNKANG J. LIU, WOODBURY, MN (US); WEI ZHANG, WOODBURY, MN (US); HERBERT C. CHIOU, CORAL SPRINGS, FL (US); BRUCE D. KLUGE, SOMERSET, WI (US); GREGORY J. ANDERSON, HUGO, MN (US)

(21) Appl. No.: 14/787,938

(22) PCT Filed: Apr. 28, 2014
(86) PCT No.: PCT/US14/35601

§ 371 Date: Oct. 29, 2015

Related U.S. Application Data
(60) Provisional application No. 61/818,090, filed on May 1, 2013.

(51) Int. Cl. A61L 15/60; A61L 15/58; A61L 15/22

(52) U.S. Cl. CPC A61L 15/60; A61L 15/22; A61L 15/58

(57) ABSTRACT

A discontinuous hydrocolloid article is disclosed that provides for a high rate of water absorption and a high rate of water vapor transmission. Also, disclosed is a method of making a discontinuous hydrocolloid article. In one embodiment, the discontinuous hydrocolloid article comprises a plurality of cross-linked polymer strands comprising a hydrophobic polymer and a hydrocolloid dispersed throughout the hydrophobic polymer and a plurality of joining strands. Each polymer strand repeatedly contacts an adjacent joining strand at bond regions.

### FIELD

The present disclosure relates to a discontinuous hydrocolloid article.

### BACKGROUND

Hydrocolloid articles are a cross-linked polymer containing a dispersion of an absorbent material. In some instances the polymer is an adhesive, which results in a hydrocolloid adhesive. A hydrocolloid adhesive can stick to a surface, while the dispersion of absorbent material is able to absorb fluid from the surface. When the absorbent material absorbs fluid the material will swell and form a gel. Therefore, unlike a foam where fluid can be squeezed out of the foam, in a hydrocolloid adhesive the fluid is held within the structure of the adhesive matrix. Also, hydrocolloids adhesives can be somewhat translucent, which allows for viewing the underlying surface without needing to fully remove the hydrocolloid adhesive from the surface. For these reasons, hydrocolloid adhesives are commonly used as medical dressings or for securing devices, such as tubing or ostomy bags, to skin.

However, hydrocolloid adhesives generally are a continuous substrate applied to the surface, such as skin. Although the hydrocolloid absorbs fluid, the rate of absorption is often slow together with very low water vapor transmission, which impacts skin adhesion and the skin conditions of the covered area.

### SUMMARY

A discontinuous hydrocolloid article is disclosed that provides for a high rate of water absorption and a high rate of water vapor transmission. Also, disclosed is a method of making a discontinuous hydrocolloid article.

In one embodiment, the discontinuous hydrocolloid article comprises a plurality of cross-linked polymer strands comprising a hydrophobic polymer and a hydrocolloid dispersed throughout the hydrophobic polymer and a plurality of joining strands. Each polymer strand repeatedly contacts an adjacent joining strand at bond regions.

In one embodiment, a medical article for contacting skin comprises a backing and a discontinuous hydrocolloid article secured to the backing. The discontinuous hydrocolloid article comprises a plurality of cross-linked polymer strands comprising a hydrophobic polymer and a hydrocolloid dispersed throughout the hydrophobic polymer, a plurality of joining strands, wherein each polymer strand repeatedly contacts an adjacent joining strand at bond regions.

In one embodiment, a discontinuous hydrocolloid article comprises a hydrophobic polymer and a hydrocolloid dispersed throughout the hydrophobic polymer and a plurality of openings in the hydrophobic and hydrocolloid dispersion. The article has a 24 hour absorption of greater than 100 wt. %, and a upright MVTR greater than 100 g/m²/24 hr and 24 hour.

In one embodiment, a discontinuous hydrocolloid article comprises a hydrophobic polymer and a hydrocolloid dispersed throughout the hydrophobic polymer and a plurality of openings, wherein the size of each opening is larger at the surfaces of the article than in the middle.

In one embodiment, a method of making a discontinuous hydrocolloid article comprises extruding a polymer strand, which comprises a hydrophobic polymer and a hydrocolloid dispersed throughout the hydrophobic polymer, at a first speed, extruding a first joining strand on a first side of the polymer strand at a second speed, extruding a second joining strand on a second side of the polymer strand, opposite the first side, at the second speed. The first speed is faster than the second speed.

In one embodiment, the polymer strands and joining strands do not substantially cross over each other. In one embodiment, a polymer strand is adjacent to a first joining strand and a second joining strand. In one embodiment, a plurality of first bond regions form between the polymer strand and the first joining strand each spaced from one another. In one embodiment, a plurality of second bond regions form between the polymer strand and the second joining strand each spaced from one another. In one embodiment, the joining strands each form a substantially straight line. In one embodiment, the plurality of polymer strands each form a wave. In one embodiment, the article further comprises an opening formed between the polymer strand and the first joining strand in an area between the successive first bonding regions. In one embodiment, the article further comprises an opening formed between the polymer strand and the second joining strand in an area between the successive second bonding regions. In one embodiment, the plurality of joining strands comprise a hydrophobic polymer and a hydrocolloid dispersed throughout the hydrophobic polymer. In one embodiment, the article further comprises a backing to which the plurality of polymer strands and joining strands are secured. In one embodiment, the backing is a woven, knitted, nonwoven, film, paper, foam. In one embodiment, the backing is coated with adhesive. In one embodiment, the backing extends beyond the polymer strands and joining strands. In one embodiment, the polymeric strands and joining strands have a circular cross-section. In one embodiment, the hydrophobic polymer comprises a hydrophobic adhesive. In one embodiment, the hydrocolloid is a water absorbing polymer. In one embodiment, the upright MVTR is greater than 100 g/m²/24 hr and 24 hour absorption of greater than 100 wt. %.

The word “strand” as used herein means an elongated filament.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments.

As used herein, “a,” “an,” “the,” “at least one,” and “one or more” are used interchangeably. The term “and/or” (if used) means one or all of the identified elements or a combination of any two or more of the identified elements.

### BRIEF DESCRIPTION OF THE DRAWINGS

FIG. 1 is a top view of a first embodiment of a discontinuous hydrocolloid article;

FIG. 2 is a top view of a second embodiment of a discontinuous hydrocolloid article;

FIG. 3 is a bottom view of a first embodiment of a medical dressing comprising a discontinuous hydrocolloid article;

FIG. 4 is a perspective view of a die comprising a plurality of orifices for making strands.

While the above-identified drawings and figures set forth embodiments of the invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of this invention. The figures may not be drawn to scale.

### DETAILED DESCRIPTION

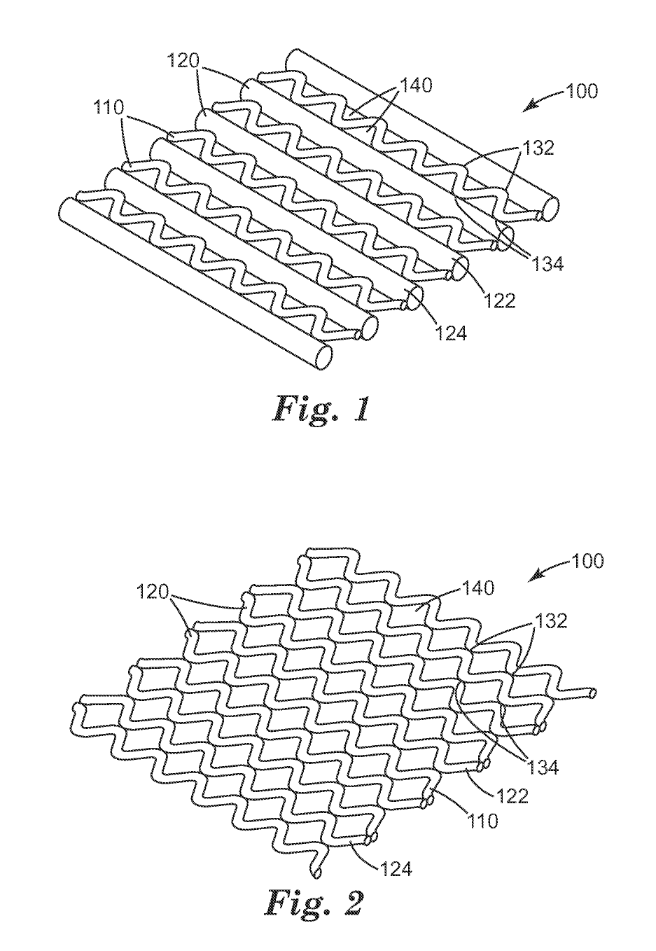

FIG. 1 is a top view of a first embodiment and FIG. 2 is a top view of a second embodiment, each showing a discontinuous hydrocolloid article 100, which comprises a plurality of polymer strands 110 and joining strands 120. A polymer strand 110 repeatedly contacts an adjacent first joining strand 122 at a various first bond regions 132, which are each successively spaced from one another. The polymer strand 110 repeatedly contacts an adjacent second joining strand 124 at various second bond regions 134, which are each successively spaced from one another. The spacing between successive first bond regions 132, and between successive second bond regions 134 forms openings 140. The openings 140 are essentially free of substance. In one embodiment, such as shown in FIGS. 1 and 2, the polymer strands 110 and joining strands 120 do not substantially cross over each other.

In one embodiment, the openings 140 form at least 5% of the total hydrocolloid article 100. In one embodiment, the openings 140 form at least 10% of the total hydrocolloid article 100. In one embodiment, the openings 140 form at least 25% of the total hydrocolloid article 100. In one embodiment, the openings 140 form less than 60% of the total hydrocolloid article 100. In one embodiment, the openings 140 form less than 40% of the total hydrocolloid article 100.

In one embodiment, the polymer strands 110 have a cross section wherein the strand 110 is widest in the middle portion and narrower at the upper and lower portion. For example, in one embodiment, the polymer strands 110 have a circular cross section. In contrast perforated structures would have a cross section with side walls in a straight line. It is believed that a cross section of the strand 110 that is widest in the middle portion increases the rate of water absorption into the hydrocolloid article 100 and allowing for increased moisture vapor transmission out of the hydrocolloid article 100. Moisture in contact with the hydrocolloid article 100 at the lower portion has more surface area to contact for increasing the rate of water absorption. Also, the narrower width at the top of the cross section of the strand also increase the surface area at the opening 140 for exiting moisture vapor. At each opening 140 the size of each opening 140 is larger at the surfaces of the article 100 than in the middle of the article 100. In other words, at a cross section an opening 140 is widest at the bottom and again at the top.

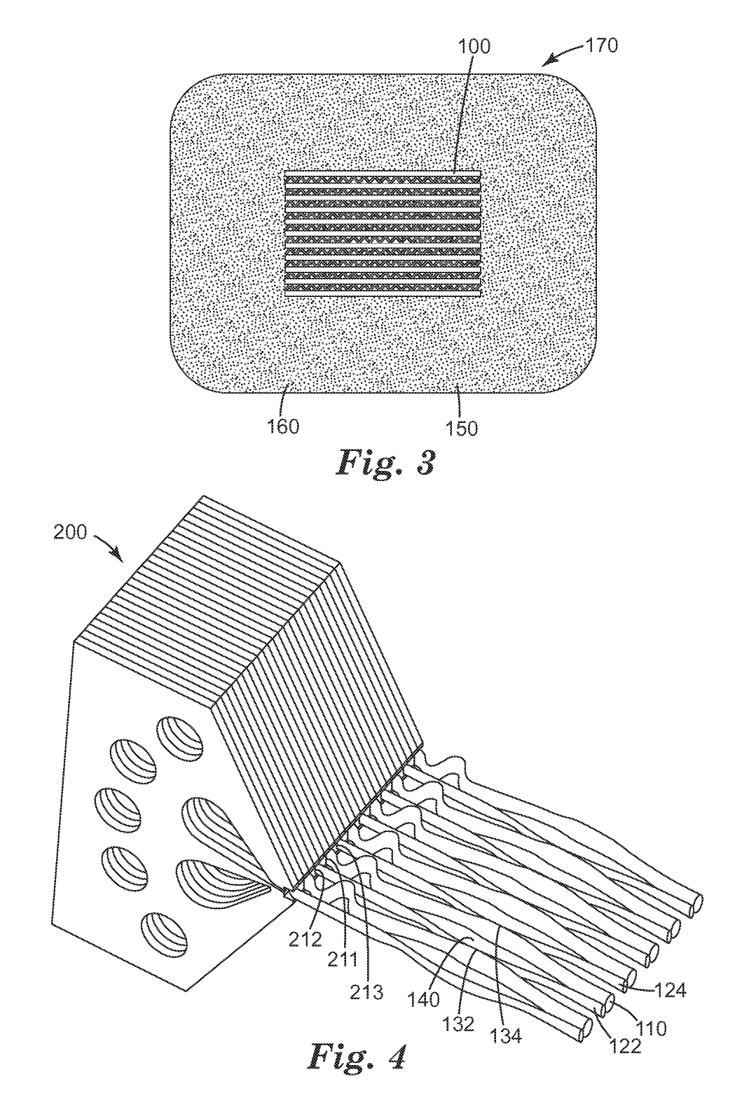

The polymer strands 110 are continuous along an x-axis, and the joining strands 120 are continuous along an x-axis (see FIGS. 1 and 2). The plurality of first bond regions 132 between the polymer strand 110 and the first joining strand 122, along with the plurality of second bond regions 134 between the polymer strand 11 and the second joining strand 124 result in the hydrocolloid article 100 having a structure that creates a barrier in the y-axis as well. Limiting fluid flow along both an x-axis and y-axis is beneficial for when the hydrocolloid article 100 (with a backing 150 applied to limit z-axis flow as well, see FIG. 3) is used on skin to limit external contaminants from entering into the covered area and to limit wound fluid from exiting the covered area.

In the embodiment of FIG. 1, the joining strands 120 are each formed in substantially straight lines, while the polymer strands 110 undulate between adjacent joining strands 120 and form a wave-like line. In the embodiment of FIG. 2, the joining strands 120 and the polymer strands 110 each undulate to form a wave-like line. Various width, dimensions, amplitude and frequency of wave for each polymer strand 110 or joining strand 120 could be used so long as the polymer strand 110 repeatedly contacts an adjacent joining strand 120, and so long as openings 140 forms between bond regions 132, 134.

In some embodiments, the hydrocolloid article 100 has a thickness greater than 0.025 mm. In one embodiment, the hydrocolloid article 100 has a thickness less than 2.54 mm. In some embodiments, the polymeric strands 110 have an average width in a range from 10 micrometers to 500 micrometers (in a range from 10 micrometers to 400 micrometers, or even 10 micrometers to 250 micrometers). In some embodiments, the joining strands 120 are of the same size as the polymeric strands 110. In some embodiment, the joining strands 120 are smaller or larger than the polymeric strands 110. In some embodiments, hydrocolloid article 100 has a basis weight in a range from 5 g/m² to 2000 g/m² (in some embodiments, 10 g/m² to 400 g/m²).

The polymer strand 110 comprises a hydrophobic polymer and a hydrocolloid material dispersed throughout the hydrophobic polymer. “Hydrophobic” means that the polymer matrix is antagonistic to, sheds, tends not to combine with, or is incapable of dissolving in water. The hydrophobic polymer may comprise blends of one or more hydrophobic polymers.

Suitable hydrophobic polymers include, but are not limited to, homopolymer or copolymer of natural or synthetic rubbers, acrylics, silicone, urethanes, acrylonitril rubber, polyurethane rubber, polyisobutylene, polyethylene-propylene rubber, polyethylene-propylene diene-modified rubber, polyisoprene, styrene-isoprene-styrene, styrene-butadiene-stryene, styrene-ethylene-propylene-stryene, and styrene-ethylene-butylene-styrene. Other, optional secondary polymers may be included in the hydrophobic polymer matrix such as elastomeric polymers or thermoplastic polymers.

In one embodiment, the hydrophobic polymer is a hydrophobic adhesive. In one embodiment, the hydrophobic polymer is cross-linked. In one embodiment, the cross-linking will be carried out by chemical crosslinking or exposure radiation, such as gamma, e-beam, or UV.

Optionally, the hydrophobic polymer can be modified with tackifying resins or plasticizers. The tackifying resins or plasticizers may or may not be miscible with the hydrophobic polymer. Useful examples of tackifying resins include but are not limited to liquid rubbers, aliphatic and aromatic hydrocarbon resins, rosin, natural resins such as dimerized or hydrogenated balsams and esterified abietic acids, polyterpenes, terpene phenolics, phenol-formaldehyde resins, and rosin esters.

Plasticizing agents can be derived from low molecular weight naphthalenic oils, or low molecular weight acids, or alcohols, which are then esterified with respectively a monofunctional alcohol or monofunctional acid. Examples of these are mineral oil, cetostearyl alcohol, cetyl alcohol, cholesterol, coconut oil, oleyl alcohol, steryl alcohol, and squalane. Some elastomers are more compatible with esters of mono- and multibasic acids, such as isopropyl myristate, isopropyl palmitate, dibutyl phthalate, diisoctyl phthalate, dibutyl adipate, dibutyl sebacate, and the like. Useful polymeric plasticizing agents include non-acrylic plasticizing agents, which are typically derived from cationically or free-radically polymerizable monomers, condensation polymerizable monomers, or ring-opening polymerizable monomers to make low molecular weight polymers. Examples of these polymeric plasticizing agents include materials such as polyurethanes, polyureas, polyvinylethers, polyethers, polyesters, and the like.

Useful plasticizing agents are compatible with the polymer(s) of the hydrophobic polymer matrix. In one embodiment, plasticizing agents are non-reactive, thus preventing copolymerization with the reactive groups of the polymers in the hydrophobic polymer matrix of the hydrophilic microparticles.

Generally, liquid plasticizing agents are readily compoundable with hydrophobic polymer matrix that includes one or more elastomers using an extruder. In addition, liquid plasticizing agents may be delivered directly to a tacky elastomer, if used in the composition, in order to make it less tacky or non-tacky.

Although somewhat more challenging to use, semi-solid (such as petrolatum) and solid plasticizing agents (such as paraffin wax, beeswax, microcrystalline wax, cetyl esters wax) can advantageously be used in compositions of the present invention where the controlled plasticization is desired. For example, hot melt processible compositions can be easily transported and handled prior to melt compounding if the hydrophobic polymer matrix and the plasticizing agent components are solid and non-tacky. Once heated to the melting or glass transition temperature of the solid plasticizing agent, the polymer of the matrix is plasticized.

In one embodiment, the plasticizing agent is used in amounts of from about 1 to 2000 parts by weight per 100 parts of the hydrophobic polymer.

The dispersed hydrocolloid material is able to absorb fluid. In one embodiment, the hydrocolloid material is a water absorbing or water swelling material. In one embodiment, the hydrocolloid material is a hydrophilic polymer. In one embodiment, the hydrocolloid material may be in the shape of a particle or a fiber. In one embodiment, the average diameter of hydrocolloid particles could range in size from 50 micrometers to 500 micrometers.

Non-limiting examples of such hydrophilic material for the hydrocolloid include: polyhydroxyalkyl acrylates and methacrylates (e.g., those prepared from 2-hydroxyethyl acrylate, 2-hydroxyethyl methacrylate, 2-hydroxypropyl acrylate, 2-hydroxypropyl methacrylate, 2,3-dihydroxypropyl methacrylate); poly(meth)acrylic acid and salts thereof (wherein (meth)acrylic acid refers to methacrylic acid and acrylic acid); polyvinyl lactams (e.g., those prepared from N-vinyl lactams such as N-vinyl-2-pyrrolidone, 5-methyl-N-vinyl-2-pyrrolidone, 5-ethyl-N-vinyl-2-pyrrolidone, 3,3-dimethyl-N-vinyl-2-pyrrolidone, 3-methyl-N-vinyl-2-pyrrolidone, 3-ethyl-N-vinyl-2-pyrrolidone, 4-methyl-N-vinyl-2-pyrrolidone, 4-ethyl-N-vinyl-2-pyrrolidone, N-vinyl-2-valerolactam, and N-vinyl-2-caprolactam); polyvinyl alcohols; polyoxyalkylenes; polyacrylamides; polystyrene sulfonates, natural or synthetically modified polysaccarides (e.g., starch, glycogen, hemicelluloses, pentosans, gelatin, celluloses, pectin, chitosan, and chitin), alginates, gums (e.g., Locust Bean, Guar, Agar, Carrageenan, Xanthan, Karaya, alginates, tragacanth, Ghatti, and Furcelleran gums), cellulosics (e.g., those prepared from methyl cellulose, hydroxypropyl methyl cellulose, carboxymethylcellulose, and hydroxypropyl cellulose); polymers prepared from water soluble amides (e.g., N-(hydroxymethyl)acrylamide and N-methacrylamide, N-(3-hydroxpropyl)acrylamide, N-(2-hydroxyethyl) methacrylamide, N-(1,1-dimethyl-3-oxabutyl)acrylamide N-[2-(dimethylamine)ethylacrylamide and -methacrylamide, N-[3-(dimethylamino)-2-hydroxylpropyllmethacrylamide, and N-[1,1-dimethyl-2-(hydroxymethyl)-3-oxabutyllacrylamide)); polymers prepared from water-soluble hydrazine derivatives (e.g., trialkylamine methacrylimide, and dimethyl-(2-hydroxypropyl)amine methacrylimide); mono-olefinic sulfonic acids and their salts, (such as sodium ethylene sulfonate, sodium styrene sulfonate and 2-acrylamideo-2-methylpropanesulfonic acid)). Other polymer include those prepared from the following monomers containing nitrogen in the non-cyclic or cyclic backbone of the monomer: 1-vinyl-imidazole, 1-vinyl-indole, 2-vinyl imidazole, 4(5)-vinyl-imidazole, 2-vinyl-l-methyl-imidazole, 5-vinyl-pyrazoline, 3-methyl-5-isopropenyl-pyrazole, 5-methylene-hydantoin, 3-vinyl-2-oxazolidone, 3-methacrylyl-2-oxazolidone, 3-methacrylyl-5-methyl-2-oxazolidone, 3-vinyl-5-methyl-2-oxazolidone, 2- and 4-vinyl-pyridine, 5-vinyl-2-methyl-pyridine, 2-vinyl-pyridine-l-oxide, 3-isopropenyl-pyridine, 2- and 4-vinyl-piperidine, 2- and 4-vinyl-quinoline, 2,4-dimethyl-6-vinyl-s-triazine, and 4-acrylyl-morpholine.

In one embodiment, 1% wt. to 65% wt. of the total hydrocolloid article comprises the dispersed hydrocolloid material. In one embodiment, from 10% wt. to 40% wt. of the total hydrocolloid article comprises the dispersed hydrocolloid material. In one embodiment, from 25% wt. to 35% wt. of the total hydrocolloid article comprises the dispersed hydrocolloid material.

The joining strand 120 may comprise a thermoplastic resin, an elastomeric material, an adhesive, a release material, or any composition of strand such as disclosed in WO 2013/032683. In one embodiment, the joining strand 120 is a hydrophobic polymer with a hydrocolloid material dispersed throughout the hydrophobic polymer. In one embodiment, the joining strand 120 is a hydrophobic polymer with a hydrocolloid material dispersed throughout the hydrophobic adhesive. In one embodiment the hydrophobic polymer is a hydrophobic adhesive.

The hydrocolloid article although comprising a hydrophobic polymer matrix, is absorbent due to the dispersed hydrocolloid material. Further, the numerous openings 140 provide flexibility, drapabality, and moisture vapor transmission from the underlying surface. Therefore, the hydrocolloid article can be used to absorb fluids on a surface. Particularly when the hydrophobic polymer is an adhesive, the hydrocolloid article can be use to both absorb fluids on a surface and to secure article to the surface. The disclosed hydrocolloid article, is especially useful for contacting skin and absorbing fluid on the skin or from a wound and allowing for moisture vapor transmission from the surface of intact skin. Further, the hydrocolloid article can be used to secure medical devices, such as tubing and ostomy bags, to skin.

In one embodiment, an additional backing 150 is included on a side of the hydrocolloid article 100. The backing 150 may be a single or multilayer structure. In some embodiments, a backing that is transparent is desirable to allow for viewing of the underlying skin or medical device. The backing 150 may comprise fabric (such as woven, knitted, nonwoven), paper, film, foam, and combinations thereof. The backing 150 may include an adhesive 160 coating to aid in securing the hydrocolloid article 100 to the backing 150. In some embodiments, the backing 150 coincides is overall size with the hydrocolloid article 100. In some embodiment, the backing 150 extends beyond the overall size of the hydrocolloid article 100, and the adhesive 160 can be further used to aid in securing to the underlying surface or skin.

The hydrocolloid article 100 may be applied directly to the backing and secure without including an additional adhesive.

In one embodiment, the backing 150 is a thin film that provides an impermeable barrier to the passage of liquids and at least some gases. In one embodiment, the backing 150 has high moisture vapor permeability, but generally impermeable to liquid water so that microbes and other contaminants are sealed out from the area under the substrate. One example of a suitable material is a high moisture vapor permeable film such as described in U.S. Pat. Nos. 3,645,835 and 4,595,001, the disclosures of which are herein incorporated by reference. In high moisture vapor permeable films or film/adhesive composites, the composite should transmit moisture vapor at a rate equal to or greater than human skin such as, for example, at a rate of at least 300 g/m² /24 hrs at 37° C./100-10% RH, or at least 700 g/m²/24 hrs at 37° C./100-10% RH, or at least 2000 g/m²/24 hrs at 37° C./100-10% RH using the inverted cup method as described in U.S. Pat. No. 4,595,001. In one embodiment, the backing 150 is an elastomeric polyurethane, polyester, or polyether block amide films. These films combine the desirable properties of resiliency, elasticity, high moisture vapor permeability, and transparency. A description of this characteristic of backing layers can be found in issued U.S. Pat. Nos. 5,088,483 and 5,160,315, the disclosures of which are hereby incorporated by reference. Commercially available examples of potentially suitable backing materials may include the thin polymeric film backings sold under the tradename TEGADERM (3M Company).

Because fluids may be actively removed from the sealed environments defined by the medical dressings, a relatively high moisture vapor permeable backing may not be required. As a result, some other potentially useful backing may include, e.g., metallocene polyolefins and SBS and SIS block copolymer materials could be used.

Regardless, however, it may be desirable that the backing be kept relatively thin to, e.g., improve conformability. For example, the backing may be formed of polymeric films with a thickness of 200 micrometers or less, or 100 micrometers or less, potentially 50 micrometers or less, or even 25 micrometers or less.

The adhesive 160 included on the backing 150 is typically a pressure sensitive adhesive. It is understood that the hydrocolloid article 100 may have sufficient adhesion to the backing 150 such that an adhesive 160 to secure with the hydrocolloid article 100 is unnecessary. However, if the backing 150 extends beyond the overall areas of the hydrocolloid article 100 an adhesive 160 on the backing 150 may be desirable, at least at the portions beyond the hydrocolloid article 100, to secure the backing 150 to the underlying substrate, i.e., skin.

Suitable adhesive for use on the backing include any adhesive that provides acceptable adhesion to skin and is acceptable for use on skin (e.g., the adhesive should preferably be non-irritating and non-sensitizing). Suitable adhesives are pressure sensitive and in certain embodiments have a relatively high moisture vapor transmission rate to allow for moisture evaporation. Suitable pressure sensitive adhesives include those based on acrylates, urethane, hydrogels, hydrocolloids, block copolymers, silicones, rubber based adhesives (including natural rubber, polyisoprene, polyisobutylene, butyl rubber etc.) as well as combinations of these adhesives. The adhesive component may contain tackifiers, plasticizers, rheology modifiers. The pressure sensitive adhesives that may be used on the backing may include adhesives that are typically applied to the skin such as the acrylate copolymers described in U.S. Pat. No. 24,906, particularly a 97:3 isooctyl acrylate: acrylamide copolymer. Another example may include a 70:15:15 isooctyl acrylate: ethyleneoxide acrylate:acrylic acid terpolymer, as described in U.S. Pat. No. 4,737,410 (Example 31). Other potentially useful adhesives are described in U.S. Pat. Nos. 3,389,827; 4,112,213; 4,310,509; and 4,323,557.

Silicone adhesive can also be used. Generally, silicone adhesives can provide suitable adhesion to skin while gently removing from skin. Suitable silicone adhesives are disclosed in PCT Publications WO2010/056541 and WO2010/056543, the disclosure of which are herein incorporate by reference.

The pressure sensitive adhesives may, in some embodiments, transmit moisture vapor at a rate greater to or equal to that of human skin. While such a characteristic can be achieved through the selection of an appropriate adhesive, it is also contemplated that other methods of achieving a high relative rate of moisture vapor transmission may be used, such as pattern coating the adhesive on the backing, as described in U.S. Pat. No. 4,595,001. Other potentially suitable pressure sensitive adhesives may include blown-micro-fiber (BMF) adhesives such as, for example, those described in U.S. Pat. No. 6,994,904.

FIG. 3 is a bottom view of a first embodiment of a medical dressing 170 comprising a discontinuous hydrocolloid article 100, such as described in FIG. 1, and a backing 150 coated with an adhesive 160. In this embodiment, the backing 150 extends beyond the overall size of the hydrocolloid article 100 so that the adhesive 160 contacts the surface, such as skin, to further secure the medical dressing 170 to the skin. The medical dressing 170 might be positioned over a wound for the hydrocolloid article 100 to absorb wound fluid. In some instances, the hydrocolloid article 100 is placed over fragile skin to protect the skin from contact with an external surface. In some embodiments, the surface of the backing opposite the surface containing the hydrocolloid article 100 includes adhesive to secure with a device, such as a medical device.

Prior art hydrocolloid articles are typically flat, continuous, planar structures and therefore can only absorb fluid in contact with the lower flat surface. In contrast, for the disclosed discontinuous hydrocolloid article 100, fluid on a surface can extend up into the openings 140 and absorb not only at the lowermost portion of the hydrocolloid article 100, but also at the inner side walls of the hydrocolloid article 100 at the openings 140. This increased surface area allows for faster absorption of fluid on the surface. For example, in one embodiment, the hydrocolloid article 100 will absorb at least 100% of its weight within 24 hours. In one embodiment, the hydrocolloid article 100 will absorb at least 200% of its weight within 24 hours. In one embodiment, the hydrocolloid article 100 will absorb at least 300% of its weight within 24 hours. In one embodiment, the hydrocolloid article 100 will absorb at least 200% of its weight within 48 hours. In one embodiment, the hydrocolloid article 100 will absorb at least 300% of its weight within 48 hours.

Additionally, the openings 140 being essentially free of the hydrocolloid article material allow for moisture vapor to pass entirely through the hydrocolloid article 100. In embodiments having a backing 150, the backing can limit the moisture vapor transmission. However, as discussed above specifically designed backing or backing/adhesive combinations can be designed to have relatively high moisture vapor transmission. In one embodiment, the hydrocolloid article 100 in combination with a backing has an moisture vapor transmission rate of at a rate of at least 300 g/m²/24 hrs at 37° C./100-10% RH, or at least 700 g/m²/24 hrs at 37° C./100-10% RH, or at least 2000 g/m²/24 hrs at 37° C./100-10% RH using the inverted cup method as described in U.S. Pat. No. 4,595,001.

Also, the openings 140 of the discontinuous hydrocolloid article 100 allow for an overall structure that is more flexible, drapable, and conformable.

The discontinuous hydrocolloid article 100 disclosed herein can be made by a process referred to a profile extrusion. For example, publication WO 2013/032683, the disclosure of which is herein incorporated by reference, discloses a profile extrusion process suitable for making the disclosed discontinuous hydrocolloid article 100.

FIG. 4 shows one embodiment of a die 200 with a plurality of orifices 211, 212, 213 for making polymer and joining strands 110, 120. Generally, the profile extrusion process comprise extrusion die including a plurality of orifices for dispensing the polymer strands 110 and joining strands 120, which are spaced from one another. In general, it has been observed that the rate of strand bonding is proportional to the extrusion speed of the faster strand. Extruder speed, orifice size, composition properties, for example, can be used to control the speed of the extruded polymer strand and joining strands.

In one embodiment, the spacing between orifices is greater than the resultant diameter of the strand after extrusion, which leads to the strands repeatedly colliding with each other to form the bond regions. If the spacing between orifices is too great the strands will not collide with each other and will not form the bond regions. Typically, the polymeric strands are extruded in the direction of gravity. This enables collinear strands to collide with each other before becoming out of alignment with each other. In some embodiments, it is desirable to extrude the strands horizontally, especially when the extrusion orifices of the first and second polymer are not collinear with each other.

In one embodiment, the polymer strand 110 is extruded from a first orifice 211 at a first speed, while a first joining strand 122 on a first side of the polymer strand 110 from a second orifice 212 and a second joining strand 124 on a second side of the polymer strand 110, opposite the first side, from a third orifice 213 both at the second speed.

In one embodiment, the extruded polymer strand 110, first joining strand 122, and second joining strand 124 do not substantially cross over each other. In one embodiment, the polymer strand 110 is oscillated between the first joining strand 122 to form the first bond region 132 and the second joining strand 124 to form the second bond region 134. Opening 140 is formed between the polymer strand 110 and the first joining strand 122 in the area between the successive first bonding regions 132 and is formed between the polymer strand 110 and the second joining strand 124 in the area between then successive second bonding regions 134.

In one embodiment, the joining strands 122, 124 each form a substantially straight line. In one embodiment, both polymer strands 110 and joining strands 122, 124 oscillate.

Typically, the orifice of the extruder is relatively small. In one embodiment the orifice is less than 50 mil (1270 micron), in one embodiment less than 30 mil (762 micron). The hydrocolloid particle used should smaller in size than the orifice opening. Generally, relatively high loadings of particles, where the particle size is not significantly smaller than the orifice size are difficult to extrude. Here, extrusion was achieved when the particle diameter ranges from 4-20 mil, and with an orifice size of 30 mil

Although specific embodiments have been shown and described herein, it is understood that these embodiments are merely illustrative of the many possible specific arrangements that can be devised. Numerous and varied other arrangements can be devised in accordance with these principles by those of ordinary skill in the art without departing from the spirit and scope of the invention. The scope of the claims should not be limited to the structures described in this application.

### EXAMPLES

Objects and advantages of this invention are further illustrated by the following examples, but the particular materials and amounts thereof recited in these examples, as well as other conditions and details, should not be construed to unduly limit this invention.

### Materials

Materials utilized for the examples and comparatives are shown in Table 1.

TABLE 1
Materials List

| Compound | Description | Source |
|---|---|---|
| Ac-Di-Sol | Croscarmellose sodium | FMC Biopolymer, Philadelphia, PA |
| CMC | CMC-PE32-FG-X, carboxymethyl cellulose | S&G Resources Inc., Medfield, MA |
| PIP | Natsyn ® 2210 polyisoprene | Goodyear Chemicals, Akron, OH |
| PIB | Oppanol ® B12 SFN polyisobutylene | BASF Corp., Florham Park, NJ |

### Test Methods

MVTR

The MVTR was determined with a method based on ASTM E96-80. Briefly, a 3.8 cm hydrocolloid laminate sample was cut and sandwiched between adhesive coated foil rings. A 118 mL glass bottle was filled with 50 mL water with a few drops of aqueous 0.2% (w/w) methylene blue. The cap for the glass bottle also contained a 3.8 cm hole. The foil ring was placed in the bottle cap and the cap was placed on the bottle with a rubber washer with a 3.6 cm opening. The bottle was placed in a 40° C., 20% relative humidity chamber in an upright position. After four hours, the bottle was removed from the chamber, sealed, and weighed (W1). The bottle was placed back in the chamber (upright position) for 24 hours at which time it was removed and reweighed (W2). The MVTR in grams of water vapor transmitted per square meter of sample area per 24 hours was calculated using the following formula.

Upright MVTR=(W1-W2)*(47,400)/24

The bottle was returned to the chamber in the upright position. After four hours, the bottle was removed from the chamber and weighed (W3). The bottle was placed back into the chamber in an inverted position for 24 hours at which time it was removed and reweighed (W4). The MVTR in grams of water vapor transmitted per square meter of sample area per 24 hours was calculated using the following formula.

Inverted MVTR=(W3-W4)*(47,400)/24

Water Absorption

A sample of hydrocolloid/polyurethane laminate was weighed, then soaked in water for 24 and 48 hours. The sample was removed from the water at the specified time and reweighed. The weight of water absorbed was divided by the initial weight of the hydrocolloid and reported as % absorption.

Adhesion

Adhesion to steel was determined with a method based on ASTM D1000. Briefly, a 2.54 cm wide by 25 cm long hydrocolloid laminate sample was applied (hydrocolloid side down) to a cleaned stainless steel plate with one pass of a 2 kg roller. An Instron tensile tester (Instron, Norwood, Mass.) was used to peel the sample at 90° at 30 cm/min. The average peel force was recorded.

### Examples

Ac-Di-Sol (12.5% w/w), CMC (15% w/w), PIP (32.5% w/w), and PIB (40% w/w) were mixed and extruded at approximately 93° C. onto a liner to produce a continuous hydrocolloid sheet approximately 0.5 mm thick. The hydrocolloid composition was removed from the liner and fed into two 3.175 cm diameter single screw extruders (length/diameter ratio of 24) at 110° C. The hydrocolloid composition was extruded through a microprofile die as shown below onto a 25 micron, corona-treated polyurethane film (Texin® resin, Bayer Material Science, Pittsburgh, Pa.) to produce a hydrocolloid laminate. The screw in the extruder feeding the polymer strands rotated at 11.3 rpm, while the screw in the extruder feeding the joining strands rotated at 8.7 rpm while. This laminate was exposed to 3 mrad e-beam radiation.

Examples 4 through 6 were prepared as described in E-1 except that the screw in the extruder feeding the polymer strands rotated at 14 rpm.

### Comparatives

Comparative 1 (C-1) was made as described for E-1, but was not exposed to e-beam radiation.

Comparative 2 (C-2) was the continuous hydrocolloid sheet described in E-1, which was laminated to a polyurethane film and exposed to approximately 40 kGy gamma irradiation.

### Results

Results for the example and comparative hydrocolloids laminates are shown in Table 2.

TABLE 2
Sample Formulations and Results

| | | | | Absorption | | |
|---|---|---|---|---|---|---|
| | e-Beam | MVTR | | (%) | | |
| | Dose | (g/24 hours/sq m) | | 24 | 48 | Adhesion |
| | (mrad) | Upright | Inverted | hour | hour | (g/2.54 cm) |
| Example | | | | | | |
| E-1 | 3 | 1238 | 1682 | 380 | 464 | 624 |
| E-2 | 4 | 1197 | 1682 | 334 | 374 | 624 |
| E-3 | 5 | 1114 | 1545 | 328 | 373 | 595 |
| E-4 | 3 | 1247 | 1730 | 378 | 441 | 567 |
| E-5 | 4 | 1238 | 1813 | 314 | 378 | 652 |
| E-6 | 5 | 1122 | 1710 | 306 | 373 | 624 |
| Comparative | | | | | | |
| C-1 | 0 | [a] | [a] | [a] | [a] | 907 |
| C-2 | [b] | 25 | 487 | 65 | 204 | 510 |

[a] Hydrocolloid debonded from the polyurethane film
[b] Gamma irradiation

1. A discontinuous hydrocolloid article comprising:
a plurality of cross-linked polymer strands comprising a hydrophobic polymer and a hydrocolloid dispersed throughout the hydrophobic polymer;
a plurality of joining strands;
wherein each polymer strand repeatedly contacts an adjacent joining strand at bond regions.

2. The discontinuous hydrocolloid article of claim 1, wherein the polymer strands and joining strands do not substantially cross over each other.

3. The discontinuous hydrocolloid article of claim 1, wherein a polymer strand is adjacent to a first joining strand and a second joining strand.

4. The discontinuous hydrocolloid article of claim 3, wherein a plurality of first bond regions form between the polymer strand and the first joining strand each spaced from one another, and wherein a plurality of second bond regions form between the polymer strand and the second joining strand each spaced from one another.

5. (canceled)

6. The discontinuous hydrocolloid article of claim 1, wherein the joining strands each form a substantially straight line, and wherein the plurality of polymer strands each form a wave.

7. (canceled)

8. The discontinuous hydrocolloid article of claim 4, further comprising an opening formed between the polymer strand and the first joining strand in an area between the successive first bonding regions, and further comprising an opening formed between the polymer strand and the second joining strand in an area between the successive second bonding regions.

9. (canceled)

10. The discontinuous hydrocolloid article of claim 1, wherein the plurality of joining strands comprise a hydrophobic polymer and a hydrocolloid dispersed throughout the hydrophobic polymer.

11. The discontinuous hydrocolloid article of claim 1, further comprising a backing to which the plurality of polymer strands and joining strands are secured.

12. The discontinuous hydrocolloid article of claim 11, wherein the backing is a woven, knitted, nonwoven, film, paper, foam.

13. The discontinuous hydrocolloid article of claim 11, wherein the backing is coated with adhesive.

14. The discontinuous hydrocolloid article of claim 11, wherein the backing extends beyond the polymer strands and joining strands.

15. The discontinuous hydrocolloid article of claim 1, wherein the polymeric strands and joining strands have a circular cross-section.

16. The discontinuous hydrocolloid article of claim 1, wherein the hydrophobic polymer comprises a hydrophobic adhesive.

17. The discontinuous hydrocolloid article of claim 1, wherein the hydrocolloid is a water absorbing polymer.

18. The discontinuous hydrocolloid article of claim 1, comprising upright MVTR greater than 100 g/m²/24 hr and 24 hour absorption of greater than 100 wt. %.

19. (canceled)

20. (canceled)

22. (canceled)

23. (canceled)

24. (canceled)

25. (canceled)

26. (canceled)

27. (canceled)

28. (canceled)

29. (canceled)
